(19) United States
(12) Patent Application Publication
(10) Pub. No.: US 2012/0053440 A1
(43) Pub. Date: Mar. 1, 2012

(54) Method for Reflex Threshold Estimation in Spastic Muscles

(75) Inventors: Matthieu K. Chardon, Chicago, IL (US); Nina L. Suresh, Chicago, IL (US); William Z. Rymer, Evanston, IL (US)

(73) Assignee: REHABILITATION INSTITUTE OF CHICAGO, Chicago, IL (US)

(21) Appl. No.: 13/222,897

(22) Filed: Aug. 31, 2011

Related U.S. Application Data
(60) Provisional application No. 61/378,685, filed on Aug. 31, 2010.

(51) Int. Cl. A61B 5/11; A61B 5/0488

(52) U.S. Cl. 600/372; 600/553

(57) ABSTRACT

A method is provided for measuring a spasticity of a muscle, comprising indenting a tapper at a particular distance into the skin, tapping a tendon associated with a muscle, and measuring the force response within a response time window. If a spastic response is detected at a particular position of the tapper according to some predefined criteria associated with the force measure, then either the position or a preload force is recorded at the first position to produce a spastic response. This determination can be used for various medical diagnoses. Furthermore, passive muscle properties can be determined by calculating a muscle stiffness value based on a line slope at at least one of the first and second indentation positions based on the maximum tapping force value at the respective indentation position.

### CROSS REFERENCE TO RELATED APPLICATIONS

The present application claims the benefit of U.S. Provisional Application No. 61/378,685, filed Aug. 31, 2010, entitled, “Method For Reflex Threshold Estimation in Spastic Muscles”, herein incorporated by reference.

### STATEMENT REGARDING FEDERALLY SPONSORED RESEARCH OR DEVELOPMENT

This work was supported in part by the National Institutes of Health Grant NIH R24 HD959721-01.

### BACKGROUND

The stretch reflex is an involuntary muscle contraction elicited by a brief stimulus to muscle receptors, such as a tap on the patellar tendon. Various medical determinations and diagnoses can be determined by a precise measurement of the stimulus threshold at which the stretch reflex (or spastic response to stimulation) occurs.

Historically, one method of determining the reflex threshold is to measure muscle reaction with an electromyographic (EMG) sensor. An EMG sensor can be used measure small electrical signals given off by muscles in the body when a change in the muscle is present. These electrical signals can thus be used to determine a point at which a spastic response is occurring in a muscle in response to a stimuli.

It is desirable to determine the precise point at which the spastic response occurs, however, and therefore, it is important to maximize the sensitivity of determining at what specific stimulus point the spasticity occurs.

Hyperactive stretch reflexes are routinely observed in stroke survivors. Research studies have shown that in stroke survivors, the stretch reflex threshold is significantly reduced in spastic muscles, and this reduction is correlated with increases in reflex joint torque. See Chardon M K, Suresh N L and Rymer W Z. A new method for reflex threshold estimation in spastic muscle. Conf Proc IEEE Eng Med Biol Soc. 1:5300-3, 2009, and Powers R K, Marder-Meyer J, Rymer W Z. Quantitative relations between hypertonia and stretch reflex threshold in spastic hemiparesis. Ann Neurol. 1988 February; 23(2):115-24.

Many clinical measures of spasticity, such as Ashworth tests and tendon tap responses, are linked to stretch reflex thresholds. In the context of estimating stretch reflex threshold, these methods are relatively imprecise and unreliable, primarily because these methods are based on qualitative assessments. Various attempts have been made to more accurately quantify a threshold for the stretch reflex, since doing so would assist in the diagnosis and treatment of various medical conditions.

Additionally, it has been desirable to determine if there are detectable physical changes in passive muscle properties associated with various medical conditions.

### SUMMARY

A system and associated method provided herein relies on a small position controlled actuator to more accurately estimate stress reflex thresholds. The use of controlled indentation of a tendon in combination with a force measurement at a point of stimulation is a practical and accurate method of estimating stretch reflex threshold as well as passive muscle properties.

The method provides for determining a precise tapping force and indentation distance at which a spastic muscle response is achieved by providing a series of taps at progressively greater indentation distances, and measuring responsive forces at a point at which the taps are delivered. Thus, one can estimate a reflex threshold in spastic muscles of patients, such as stroke survivors, using controlled amplitude taps superimposed on progressive muscle indentation of tendons, such as the bicipital tendon in the bicipital fossa, where the threshold is determined based on a measure of force determined at the point of tap application. This muscle indentation can be done with, e.g., a linear actuator positioned over the muscle tendon at a specific location on the body, such as at the elbow for the bicipital tendon in the bicipital fossa.

In accordance with various embodiments of the invention, both reflex force and EMG responses to the pulses are elicited at much lower initial loads on the spastic side as compared to the contralateral side, however the stimulus force is similar between the two sides. The use of controlled indentation of the tendon is therefore a practical and accurate method of estimating the stretch reflex threshold as well as passive muscle properties and thereby quantifying spasticity in a simple, rapid and cost effective manner.

Additionally, in certain situations, physical changes in passive muscle properties can be detected according to the force measurement techniques described above, that are indicative of a particular medical condition.

Accordingly, a method is provided for measuring a spasticity of a muscle, comprising: a) generally immobilizing a muscle; b) placing a tapping element at a skin position over a tendon associated with the muscle to establish a tap point; c) moving the tapping element to an indentation position at a predetermined distance from a reference point; d) at the indentation position, measuring a preload force on the tapping element and then providing one or more taps in a tap group at the tap point; e) measuring a force curve on the tapping element during a predefined reflex response time window after providing each said tap; f) determining and recording, for each said tap, a tap response value based upon a predefined tap criteria associated with the force curve; g) determining, for each said tap group, whether or not a spastic response is present based upon a predefined tap group criteria associated with the tap response values within at least one of a current or previous tap group; h1) if no spastic response is determined to be present, then moving the tapping element to a new indentation position and repeating steps (d)-(g); and h2) if a spastic response is determined to be present, then establishing at least one of the indentation position and the preload force as the determined muscle spasticity threshold.

Furthermore, a method for measuring a stiffness of a muscle, comprising: a) generally immobilizing a muscle; b) placing a tapping element at a skin position over a tendon associated with the muscle to establish a tap point; c) moving the tapping element to a first indentation position; d) at the indentation position, measuring a preload force on the tapping element and then providing one or more taps at the tap point; e) measuring forces on the tapping element prior to a reflex response time window after providing the tap(s) and determining a maximum value that is a maximum tapping force value; f) moving to a second indentation position, and repeating steps (d) and (e); and g) calculating a muscle stiffness value based on a line slope at at least one of the first and second indentation positions based on the maximum tapping force value at the respective indentation position.

### DESCRIPTION OF THE DRAWINGS

Various embodiments are disclosed in the following drawings.

FIG. 1 is a pictorial diagram illustrating an experimental set up of a position-controlled linear actuator placed perpendicular to the tendon of the biceps brachii;

FIG. 2A is a flowchart illustrating the spastic threshold detection and measurement process;

FIG. 2B is a flowchart illustrating the passive muscle properties measurement process;

FIG. 2C is a graph showing an exemplary tapping sequence;

FIG. 2D is a graph showing a series of tapping sequences applied at different positions corresponding to tendon depth;

FIG. 3A is a graph illustrating a typical force trace versus time and the trace's constituent components;

FIG. 3B is a graph illustrating a rectified surface EMG signal;

FIG. 3C is a graph illustrating the reflex threshold and its standard deviation for a given subject using the permutation technique;

FIG. 3D is a graph illustrating the reflex threshold using the piecewise comparison technique;

FIG. 3E is a graph that is a zoom of FIG. 3D showing the reflex threshold being determined by comparing the values in the solid ellipse to the values in each of the doted ellipses;

FIG. 4 contains graphs illustrating different force v. position measurements for the spastic and contralateral muscles for investigating the reflex threshold; and

FIGS. 5A & B contain graphs illustrating different force v. position measurements for the spastic and contralateral muscles for investigating passive properties of the muscle.

### DETAILED DESCRIPTION

Various embodiments of the invention are demonstrated and described below in the context of various experiments conducted.

### Embodiment 1—Threshold Detection

### Experimental Setup for Force Measurement

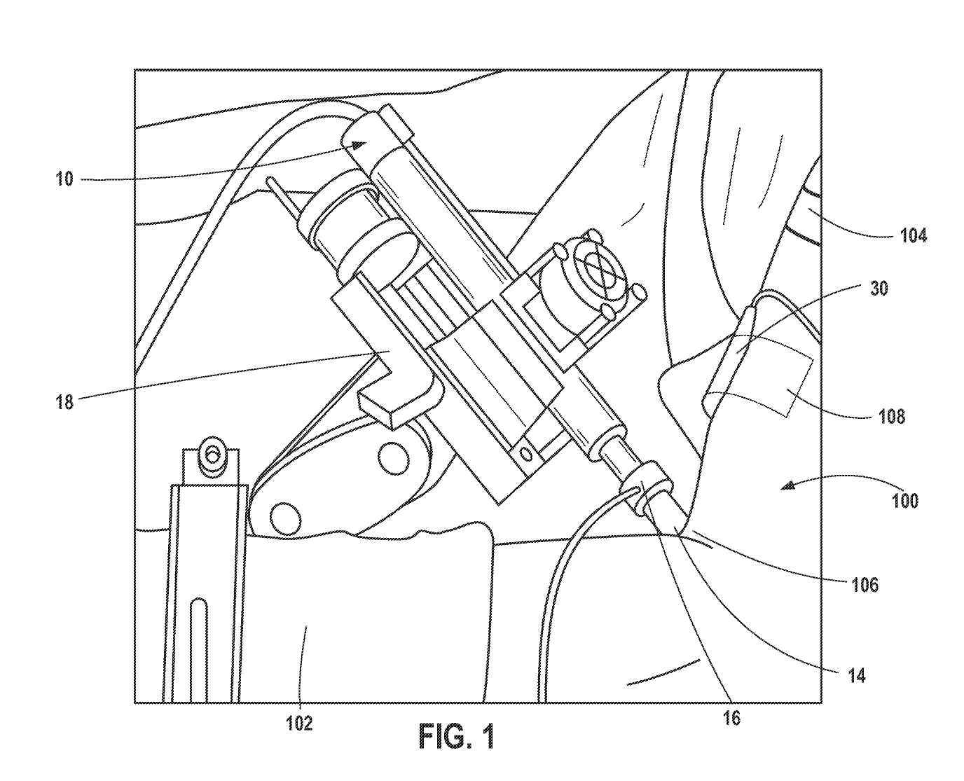

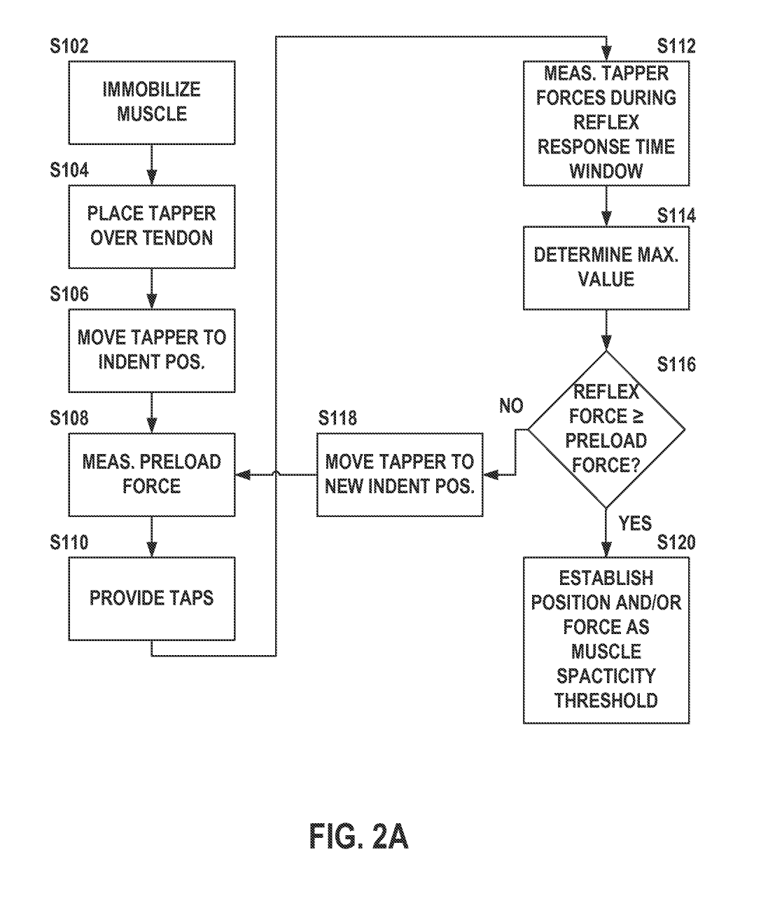

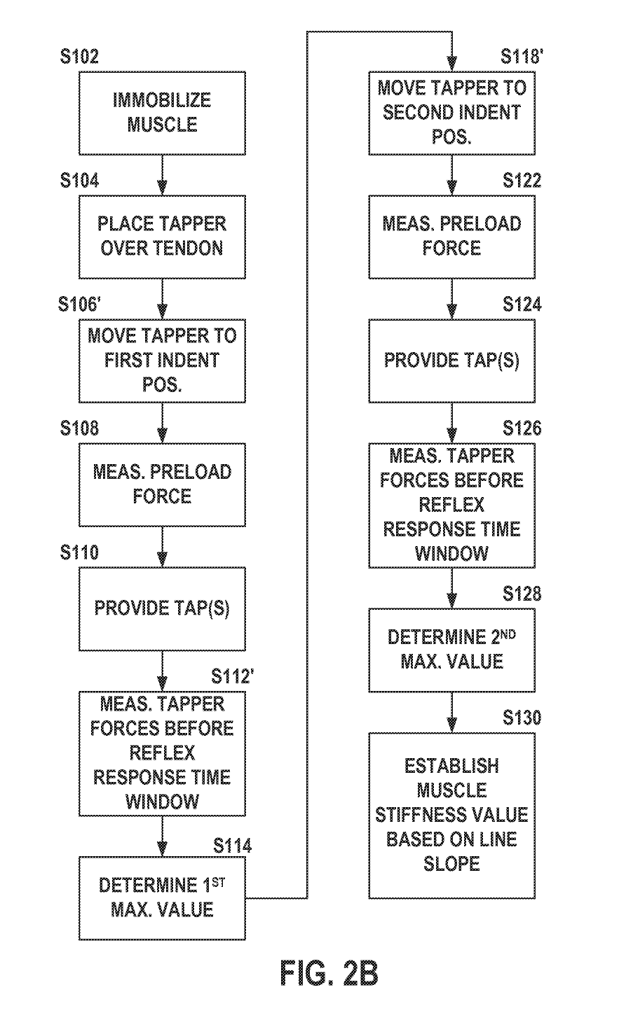

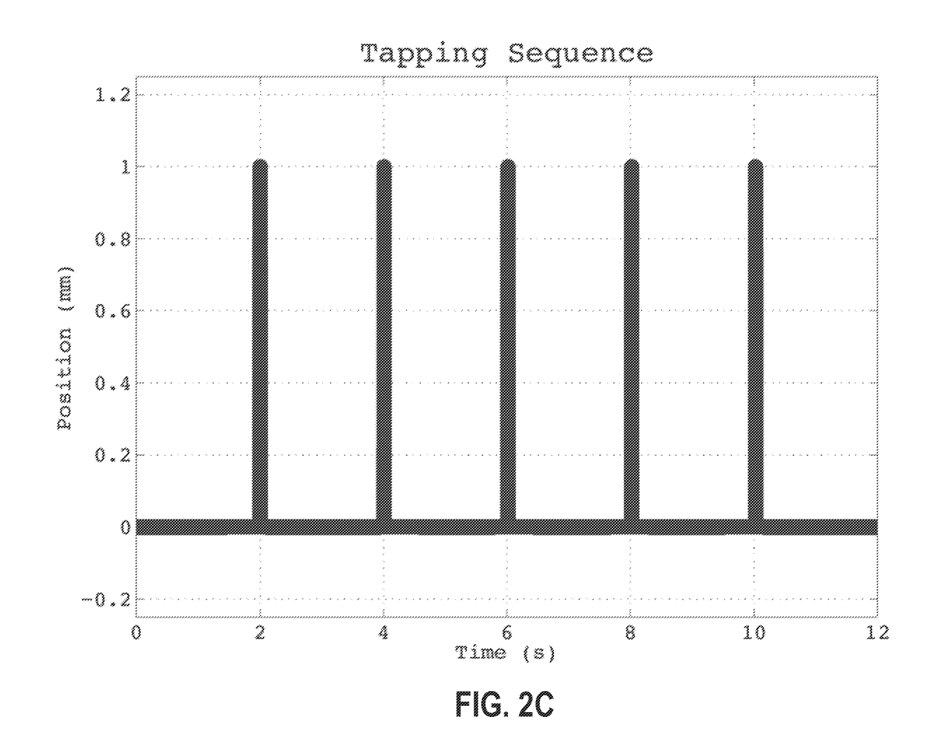

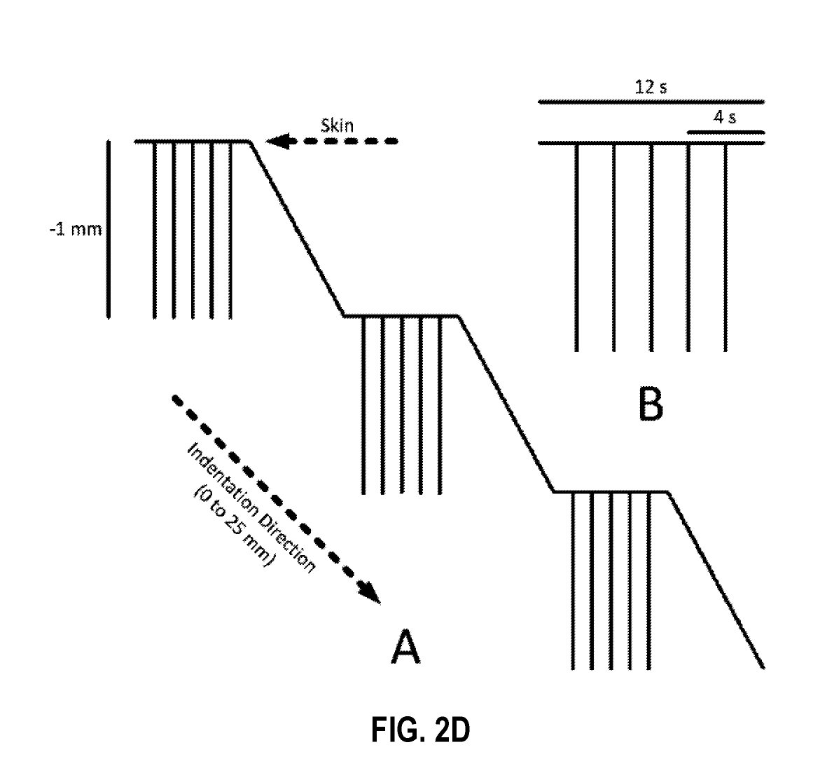

FIG. 1 is a pictorial illustration of an exemplary setup used to demonstrate a system that relies on a small actuator to accurately estimate the stretch reflex threshold, using small amplitude dynamic muscle stretches superimposed upon a progressively increasing muscle pre-load in a passive muscle. FIG. 2A is a flowchart illustrating the process that was followed.

In the exemplary setup shown in FIG. 1, the reflex threshold estimates in the passive spastic and contralateral elbow flexor muscles of four hemiparetic spastic stroke survivors were compared, using a position-controlled linear actuator 10 (manufactured by Linmot, Inc.) to apply controlled indentations of the tendon of the biceps brachii 106.

In order to utilize tendon tension as a measure of the reflex response, an isometric condition was established using a mechanical frame to stabilize the limb. Subjects were strapped to a chair, with the arm and wrist casted, and clamped on a magnetic base, assuring the relative immobility of the elbow joint (FIG. 1). The limb 100 (FIG. 1) was fully stabilized S102 (FIG. 2A) by securing the upper arm 104 and wrist (cast) 102 to a workbench.

The arm position was optimized such that the elbow and shoulder joint angles ensured a maximal reflex response from the biceps. In general, the position should provide an optimum tendon length vs. bone. For example, for the biceps, an ideal subject position is approximately 45° wrist supination, a 120° elbow extension, a 45° shoulder abduction and a 10° shoulder flexion. Subjects were placed as closely as possible to the ideal joint angles. These angles were recorded for all subjects to ensure repeatability on the contralateral side. Clearly, however, these precise positions do not reflect critical parameters and are not required to provide accurate results—other positions may be utilized and the parameters can be varied, depending on other factors.

A Linmot position controlled linear actuator 10 was mounted S104 on the mechanical frame directly above the patients' tendon 106 and used to elicit the reflex response. The linear actuator 10 was placed at the tendon of the biceps brachii at an orientation of approximately 90° to the long axis of the biceps brachii (a preferred, but not essential, orientation) and the position of the tapper tip 14 was visually zeroed at the surface of the skin. The tapper tip 14 was placed on the biceps brachii tendon approximately 75-80 mm to the humerus.

The tapper tip 14 was lowered onto the tendon to an indent position S106 relative to the skin. Further iterations of the following process were performed based on different indent positions. In the experiment, 1 mm indent increments were used, and accurately measured using a micrometer 18 attached to the actuator 10. The position of the linear actuator 10 was constantly monitored, guaranteeing repeatability in the stimuli and in the measurements of the reflex.

Once the tapper tip 14 was lowered to an indent position, a preload force was measured S108. A force sensor 16 made it possible to measure the tension in the tendon at rest and during a reflex response, using a high precision load cell mounted between the linear actuator 10 and a rubber bumper that interfaced with the patients' skin. The force sensor 16 was implemented by a Sensotec single axis load cell that recorded the static preload force applied to the tendon, the stimulus force (i.e., tap force), as well as the (dynamic) load produced by the reflex response at the point of application. Although this was a preferred configuration for the experiment, other force-measuring elements could be utilized. The static preload was used as a marker for tendon (and muscle stretch) relying on the assumption that tendon stress is linearly related to tendon strain, for small perturbations.

Once the preload force was measured, the actuator 10 was then used to tap the tendon according to a predefined tap sequence S110. Two measures were used to quantify the reflex responses: 1) the force trace measurement of the tension in stimulated muscles tendon; and 2) the EMG activity of that muscle (discussed in more detail below). Both measurements provided a benchmark for the reflex threshold estimations and required different methods. In the experiment, surface EMG of the biceps brachii and triceps was recorded using an EMG electrode 30.

At each position, five transient taps in a tap group indented the tendon for 1 mm in 1 ms, at two second intervals (FIG. 2C). The taps were administered over a distance of 25 mm indentation into the tendon in 1 mm steps for each tap group (FIG. 2B). The use of tap groups instead of individual taps at each indentation position permits a more stable measurement. The use of an odd number of taps in a tap group permits some form of a majority-based decision to be made (i.e., some majority of taps in the tap group meet some predefined criteria). The use of five taps in a group has been found to be a good practical number, permitting reliable variance figures to be determined, and providing a good balance between accuracy and time. The techniques described herein are not limited to five taps in a tap group and could apply to any number of taps in a tap group. A tap “group” of only one tap could be utilized as well (in which case the determination of a response from the tap group constitutes the determination of a response for an individual tap), although the values obtained may not be as accurate.

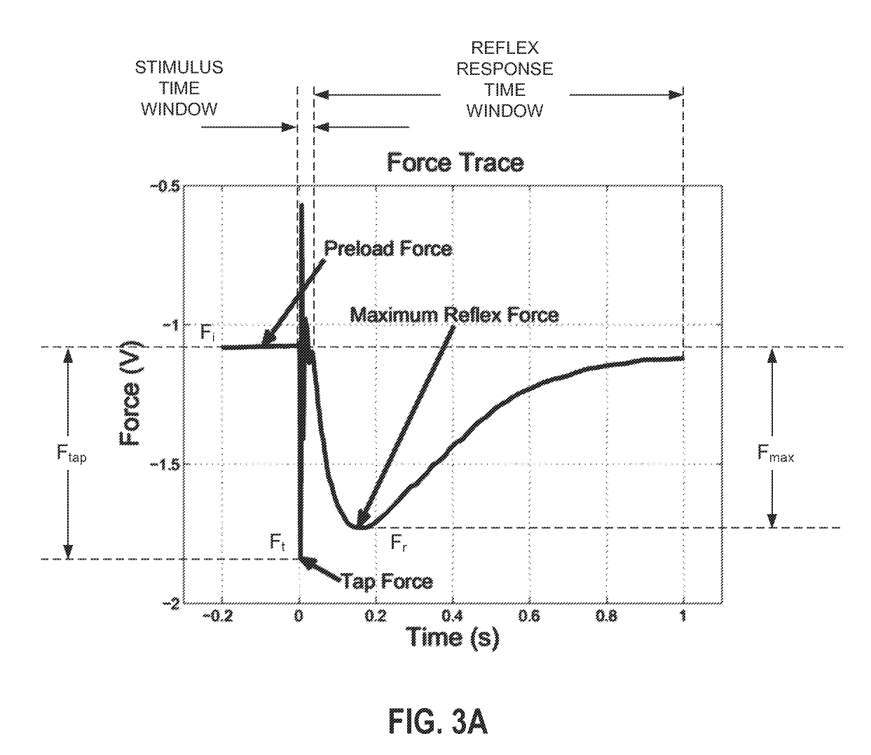

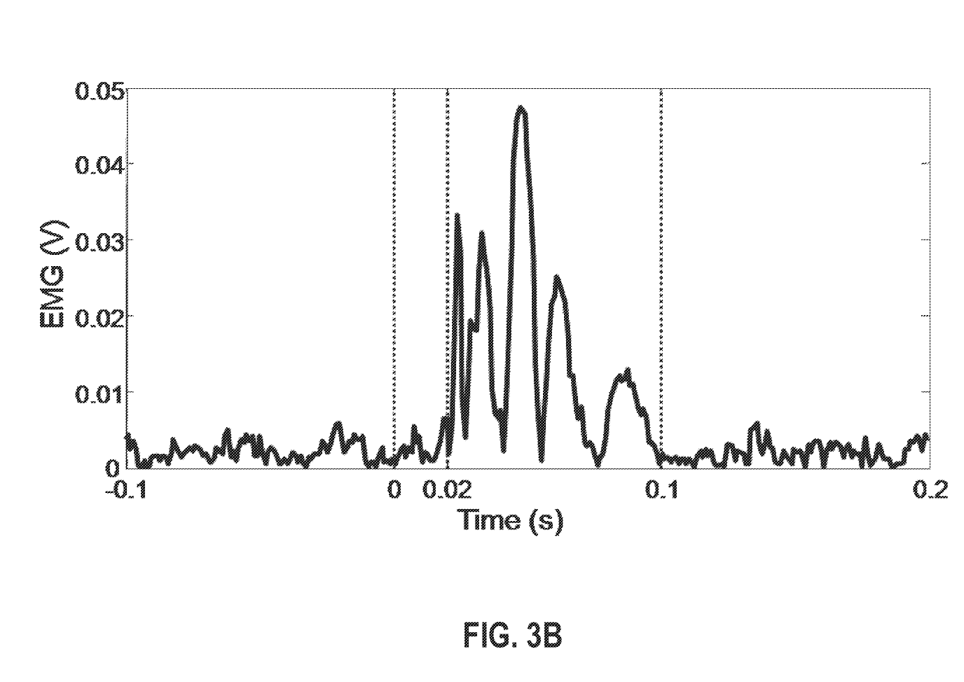

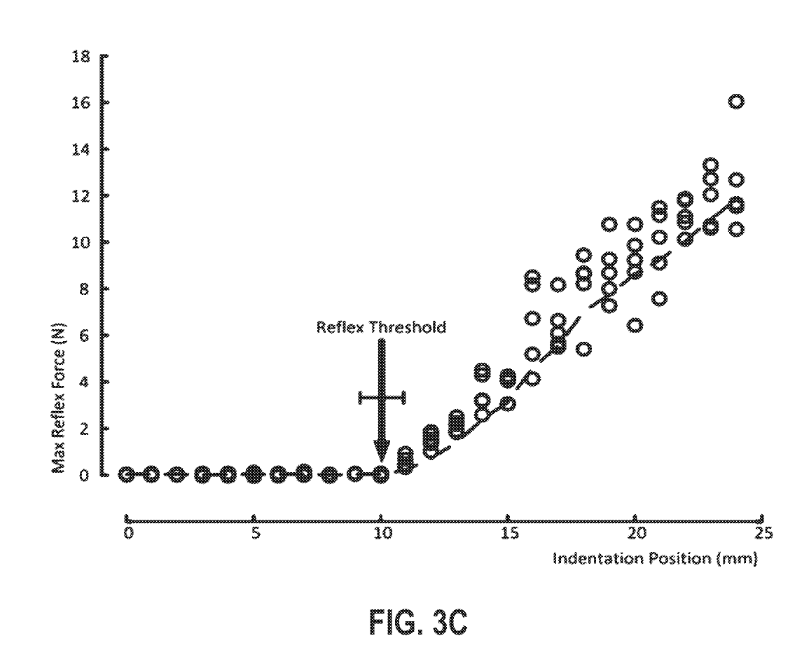

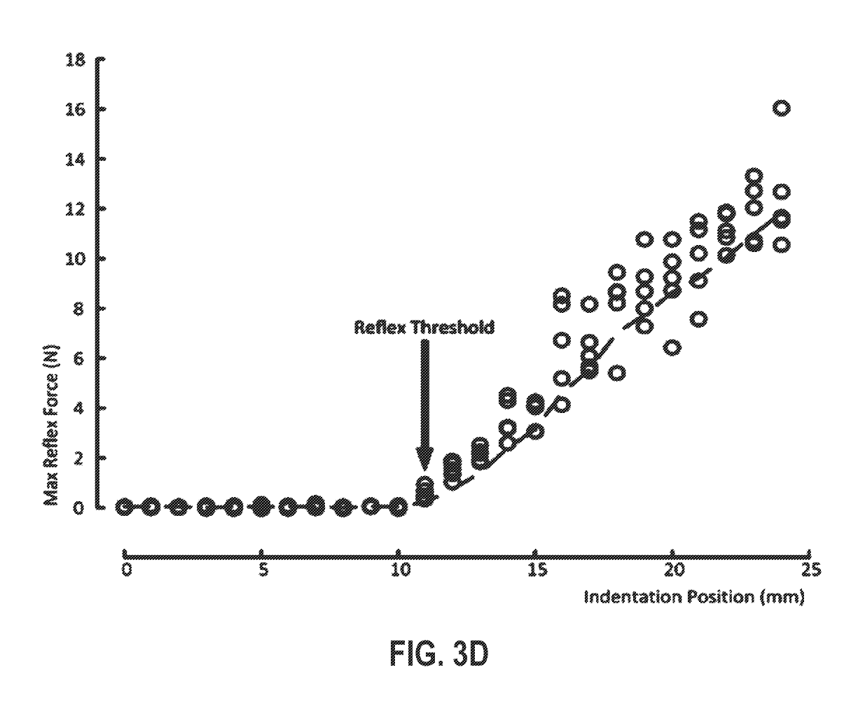

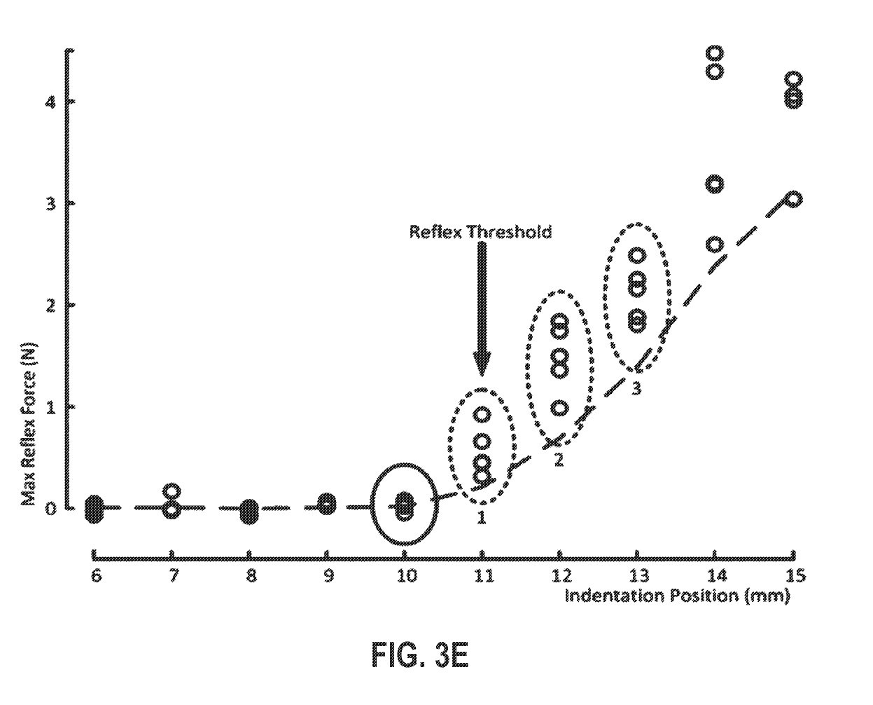

A force curve was measured after each transient tap during the reflex response time window S112. FIG. 3A shows the force trace as a continuous resultant force of the actuator on the tendon. As noted above, three different pieces of information were extracted from the trace: a) the preload force, which is the force at which the tendon is initially loaded—it is the average static force or pressure on the tendon prior to the stimulation, b) the tap force, which is the force exerted by the delivered tap relative to the preload, and c) the maximum reflex force which is the maximum measurement delivered by a reflex response, i.e., the maximum force generated by the muscle in response to the tap, relative to the preload.

Two parameters were extracted from the recorded force trace shown in FIG. 3A: 1) the maximum tap force and 2) the maximum reflex response S114. The maximum tap force was calculated as follows:

Fₜₐₚ=Fₜ−Fᵢ, (1)

where:
Fᵢ is the pre-load force on the tendon;
Fₜ is the absolute force applied on the tendon by the transient tap; and
Fₜₐₚ is the relative force between the absolute force applied on the tendon by the transient tap Fₜ and the pre-load force on the tendon Fᵢ (FIG. 3A).

Fₜₐₚ is interpreted as the passive visco-elastic properties on the muscle.

The second value is the maximum reflex force which is calculated as follows:

Fₘₐₓ=Fᵣ=Fᵢ, (2)

where:
Fᵣ is the absolute reflex force; and
Fₘₐₓ is the relative force between absolute reflex force Fᵣ and the pre-stretch force on the tendon Fᵢ (FIG. 3A).

In one test in which a preload position of 2 mm was used, a stimulus was given at time 0 for 2 ms with resultant forces from the stimulus itself lasting approximately 20 ms. The reflex response started roughly at 20 ms (this may vary, depending on size of subject—tables may be utilized to store values for response start and stop times based on predefined values or within-subject/across-subject cumulative averages). The force generated after the 20 ms represents the reflex force of the biceps muscle. This force reached an apex at 130 ms and this apex value is defined as the maximum reflex force.

To estimate reflex threshold, successive position changes of the tendon were then imposed using muscle indentation that was measured using the micrometer 18. The stretch reflex response (both EMG and force) to five successive pulses (see FIG. 2C) at each indentation position (see FIG. 2D) were recorded. According to the experiment indentation position increments of 1 mm were utilized for performing the tap groups, although other increment sizes could be utilized as well. If the arm and muscle are immobilized (i.e., no joint movement), as done in this setup, the change in indentation position produces a measurable change in the tension of the tendon attached to the muscle.

At each tap at a particular indentation position, the spasticity threshold was determined to have been met when a predefined criteria was met. One such predefined criteria is where the maximum value of the measured forces during the reflex response time window (Fᵣ) is greater than or equal to the preload force (Fᵢ) by some amount over a noise level.

The spasticity threshold can be determined with both position and preload force. As far as the force measurement is concerned, Fₘₐₓ is the preferred measure used to detect threshold. This value must be greater than zero to detect it, but the threshold is at the lowest value of Fₘₐₓ that is above zero (and above noise levels). Finally, if Fᵢ vs Fₘₐₓ is plotted, rather than position vs. Fₘₐₓ then the Fᵢ at which any measurably detectable reflex activity is considered to be the threshold. In other words, position may be used as a threshold value or Fᵢ, depending on what the controlled variable is.

Sample data from the force response curve can be stored, and additional values can be calculated for potential diagnosis use. For example, the response rise times and other curve properties can be calculated and utilized for various diagnoses and procedures.

For each tap it was determined if there was a response from the muscle by comparing the Mean Max Reflex Force to the Mean Preload Force (based on sample measured values) plus three times its standard deviation. If the Mean Max Reflex Force was larger than the Mean Preload plus three times its standard deviation, the response was considered to be a valid muscle response. The Mean Max Reflex Force can be determined in a number of ways. One accurate way of making this determination is to find a peak value in the response curve, and performing a summation on sample values on either side of this peak value within some time window (e.g., 20 ms on either side of the peak time). Of course, a summation could also be made over other windows of time for the response curve.

The use of determining a response if the Mean Max Reflex Force was larger than the Mean Preload plus three times its standard deviation is a way of determining if the response curve is above a noise-level threshold. The key value to determine is, “is the measured response curve above the noise threshold?”. The three times standard deviation is a good way of making this determination, but it is not the only way, and other techniques could be used to separate actual responses from noise.

In the experiment, and according to an embodiment of the invention, a binary value, 0 for a non-response or 1 for a response was assigned to a particular tap. These results were placed into a “Response Matrix” with five rows representing each tap and with each column representing the indentation position.

### Reflex Threshold Estimation

Three different techniques were considered for finding the reflex threshold estimation. The first uses a permutation technique, the second uses piecewise comparisons, while the third fits curves.

The first technique uses the permutation method. The reflex threshold is obtained by permuting the Response Matrix, calculated above, along the indentation direction. By way of example, presume the response from five taps for twenty-four indentation positions were recorded. The Response Matrix would be a 5×24 Matrix filled with binary values (0s or 1s). This matrix would be permutated along the indentation position such that the first row of the “Permutated Matrix” would be filled with binary value of the first tap of each indentation. The second row would be then filled with the first tap for the initial indentation, the second tap for the second indentation and the first tap for the rest of the indentation. This process would be repeated until the Permutated Matrix would be filled with 24⁵ by 24 possible combinations.

The Permutated Matrix would therefore be filled with all possible combinations of binary values along the indentation position. The threshold in this technique is found by finding, for each permutation, the first indentation position at which there are three consecutive positive reflexes in a row. That is finding for each permutation the first indentation position that has three 1s in a row. Overall we have 24̂5 thresholds, and their mean becomes the Spastic Reflex Threshold for the subject. FIG. 3C illustrates the reflex threshold and its standard deviation for a given subject using the permutation technique at 10 mm with a standard deviation of ±1 mm. The permutation method assumes that the order of the taps does not affect the results.

The second technique uses piecewise comparison of sequential indentation data. The main idea behind this technique is following: each indentation level is compared to its neighbor until a difference is detected. The method thus walks its way down the indentation level comparing five indentation response groups given by each tap. Sometimes there are no responses and sometime there are responses (FIG. 3D).

To determine if a reflex threshold has been reached, contiguous indentation responses are compared. For example, the five responses at indentation position 10 mm will be compared to the five responses at indentation position 11 mm (FIG. 3E, which is a zoom of FIG. 3D). If the five responses are different from each other, then the comparison is done between the indentation position 10 mm and indentation position 12 mm. This is repeated until three indentation positions in a row have been shown to be different from the initial one (solid ellipse in FIG. 3E). The Reflex Threshold is marked by the red arrow. The reflex threshold is determined by comparing the values in the solid ellipse to the values in each of the doted ellipses.

To perform the comparison between the each group of five responses, different statistical techniques can be used, such as the t-test and the Kruskal-Wallis test. In the experiment, each of these tests was set to a significance of 0.05.

The final technique used to find the reflex threshold incrementally fits two linear curves to the data. Each of the two fits are incrementally given sets of responses, one starting from the left and the other starting from the right. As more and more data is given, the better the fit gets for each of the curves. This trend persists until the fits start to decline. This decline marks the position of the reflex threshold.

If the threshold was determined S116 to have been met, then either the indentation position or the force was recorded as the muscle spasticity threshold S120. If the threshold was determined to have not been met, then the tapper was moved to the new indentation position S118, and the preload forces were measured again S108, the tap groups provided S110, force measurements taken S112, the maximum value was determined S114, and the threshold test S116 once again performed.

Each position was treated as a new trial. In some subjects, the number of trials was limited on the affected side by the large force generated by the reflex response, which could shut the actuator controller off. The controllability of the linear actuator insured the repeatability of the stimuli over the duration of each experimental session. Additionally, since the actuator was placed so as to pre-stretch the tendon, constant contact between the tendon and the actuator was ensured.

### Experimental Setup for EMG Measurement

The second measure used was EMG. Three active DELSYS electrodes 30 were placed on the medial and lateral heads of the biceps brachii muscle 108 at about the center of the muscle (i.e., half way between anatomical landmarks at the shoulder and the elbow), and one on the triceps brachii (not shown). The study examined the stretch reflexes of the biceps brachii in both sides of four hemi-spastic stroke individuals. A clinical assessment was performed by a physical/occupational therapist, upper arm impairment was assessed using the Fugl-Meyer test and the Chedoke-McMaster assessment.

The DELSYS preamplifier has a bandwidth of 20-450 Hz for surface recordings with a gain set at 100× for all tested subjects. The surface EMG signal was digitized at a rate of 2 kHz (CED, Power 1401). Honeywell Sensotec-measured force or force applied/generated at the biceps tendon was filtered with a bandwidth of 500 Hz and sampled at a rate of 1000 Hz. The data was collected using a software program from Cambridge Electronic Design (CED), Spike2 (version 6), and stored on a computer for subsequent analysis.

FIG. 3B is a graph of an exemplary rectified EMG signal where the stimulus was given at time 0 for 2 ms with direct effects from the stimulus lasting approximately 20 ms. After 20 ms, the electrical activity of the muscle was measured for the next 80 ms or so. However, unlike the force trace, the EMG signal dies down after approximately 100 ms.

To quantify the surface EMG activity of the biceps, the average rectified EMG (RIEMG) was computed as follows:

$$RIEMG = \frac{\sum V - \sum {V}_{pre}}{\sum {V}_{pre}} \qquad ( 3 )$$

where
V is the voltage of the reflex; and
Vₚᵣₑ is the voltage prior to a stimulation.
   In the above equation, it is understood that the summation windows are equivalent for both V and Vₚᵣₑ. The summation window is equivalent to the integral bounds in a continuous system. For example, one would sum all the values from 0 s to 4 s which would be a 4 s window. In the present case, one would sum the same amount of values for Vₚᵣₑ and V. The calculated measured RIEMG value is compared against a threshold RIEMG value to establish that a spastic response has taken place. Threshold RIEMG values can be established for a particular sensor system, muscle, and subject attribute, such as size. As with the predetermined force values, the threshold RIEMG values can be based on averages/medians of within-subject or across-subject accumulations of data values.

### Results

The above-described tendon tapping method made it possible to detect distinct EMG and force threshold differences between the affected and contralateral side of all four tested stroke subjects (Table I). Further, the threshold values derived from the EMG and force traces in the same subject were consistent with each other (Table I).

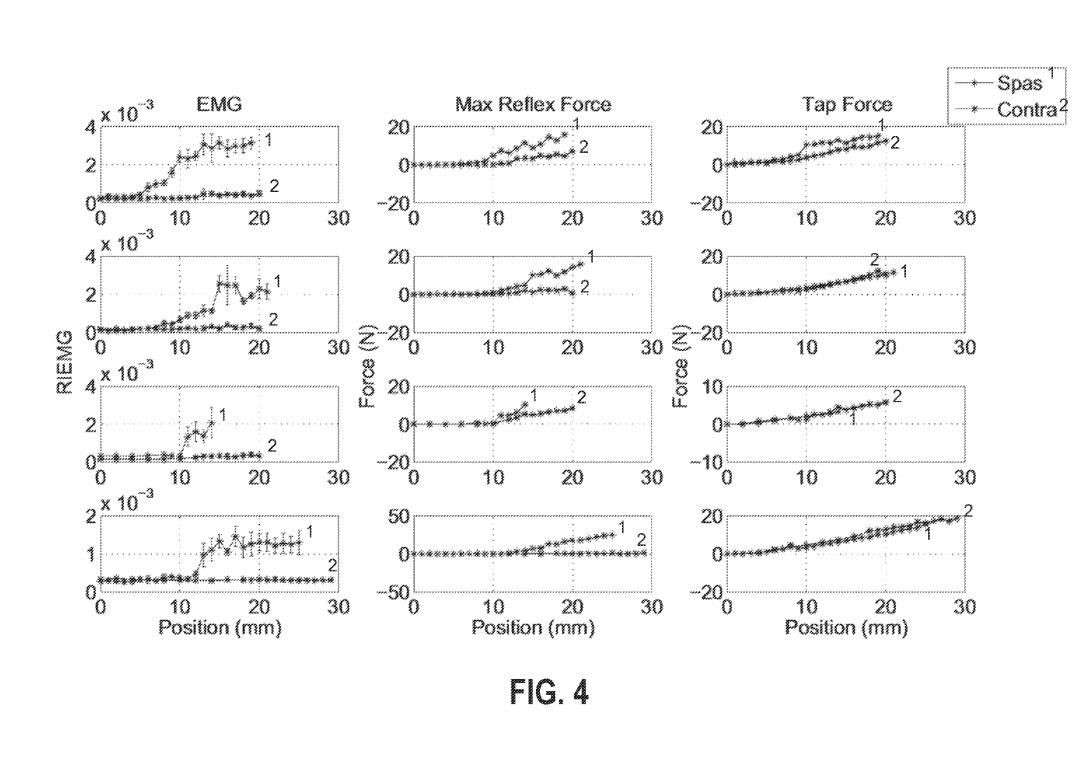

In three of the four subjects, the EMG recording showed that the threshold of the stretch reflex was markedly lower for the spastic side as compared to the contralateral side (FIG. 4 and Table I).

TABLE I
Indentation Position of Threshold for Each Subject

| | |
|---|---|
| | Max Reflex |
| EMG (mm) | Force (mm) |

| Subject | Spastic | Contralateral | Spastic | Contralateral |
|---|---|---|---|---|
| 1 | 5 | 13 | 6 | 13 |
| 2 | 7 | 14 | 9 | 14 |
| 3 | 10 | 11 | 10 | 11 |
| 4 | 12 | — | 12 | — |

This technique allows obtaining two fundamental values for spasticity. The first is the THRESHOLD at which the taps produce any measurable reflex force within the reflex response time window. The other is the GAIN of the reflex which is obtained as indentation is continued beyond the threshold point. Gain relates position to max force and it is the slope of the curve. The gain thus relates to the muscle delivering more force for a given indentation position. A high gain could, e.g., infer that more motor units are recruited for the same indentation.

Since data was collected over a fairly large range of tendon indentations, it was possible to quantify and characterize the relation between input tap force and indentation, a relation that was approximately linear (FIG. 4). It was also possible to characterize the relation between the reflex force and tendon indentation, which was also approximately linear. In contrast, the relationship between EMG and tendon indentation was approximately sigmoidal in shape (FIG. 4).

In three of the four subjects, the forces elicited by the taps were identical on the two sides over the range of tested indentations. In the fourth subject, the tap force was similar over a large part of the range. When tapping force was plotted against tapper indentation position, the relationship was linear in all four of our subjects. The tap force is therefore also a measure of the passive properties of the musculotendinous apparatus, and this constancy shows that for matched initial tap responses, comparable stimuli is delivered to the tendons on both sides of the subject.

This method advantageously permits easy separation of tendon mechanical force responses from reflex force responses. This is because the stimulus time window does not overlap the time of the reflex response (FIGS. 3A, B).

Importantly, a linear position controlled actuator can be used to indent the tendon of spastic muscle, as a way to produce controlled muscles stretch, without moving the whole limb. When tendon force is used as a surrogate measure of stretch, and tendon taps are superimposed to generate responses, the threshold muscle lengths were systematically longer in spastic as compared with contralateral or normal control muscles.

There are many potential benefits to this tendon loading/tapping technique. First, the input to the system is controlled and repeatable, making it possible to set a base line level of stretch and to stimulate the system with a broad range of input amplitudes and bandwidths. Second, unlike EMG measurements, which are often non-linear, tendon strain measurements are able to quantify the activity of the muscle as a whole in a relatively linear manner. Third, because one is able to obtain input force measures, the force trace provides information on reflex activity and also on the passive muscle properties. Finally, in the spectrum of reflex stimulators, the tendon tapper disclosed herein can be made more compactly and lower in cost than large-scale actuated systems used to manipulate whole limbs. In order to reliably elicit and measure a reflex with the device, the joint simply has to be immobilized and the device accurately placed on the tendon.

The use of force measurements is preferable over EMG measurements. FIG. 4 illustrates a series of graphs reflecting tap sequences that were administered on both sides of each individual. The spastic side measurements are represented by the curve designated “1”, and the contralateral side measurements are represented by the curve designated “2”. For the first two columns (EMG & Max Reflex Force) a clear difference can be seen between the spastic and contralateral side with regards to EMG activity and reflex force suggesting a difference in reflex susceptibility.

For the third column there appears to be no clear difference, suggesting similar passive viscoelastic properties for both sides. By plotting position vs Fₜₐₚ of both limbs on the same graph, on can compare their intrinsic stiffness. If their intrinsic stiffness is the same, then one can conclude that both sides have the same visco-elastic properties. However, in some subjects, discussed in more detail below, some patients had differences, suggesting that the internal structure of their muscle were different on both sides.

Both the EMG and the force measurements are able to show the existence of a reflex threshold (FIG. 4). However, as the tap force is increased incrementally, the RIEMG values often reach a plateau, where the maximum reflex force keeps increasing, suggesting saturation in the EMG measurements. This phenomenon may be due in part to the sampling properties of the EMG electrode, which can only record electrical activity across a given surface area. At higher muscle activation levels, the EMG measurements underestimate the activity of a muscle and subsequently the motoneuron pool. It also becomes difficult to quantify the system when the relationship between the input and output is non-linear, as compared with steady force measurements.

The threshold at which the motor pool responds to a stimulus can be estimated using both EMG and tendon tension measurements. For spastic data, the threshold is clearly discernible, and it can be detected by eye. For the control, one must rely on mathematical detection methods. In the present case, two standard deviations from the mean were used as a criteria to determine the threshold. The force trace is more suited to determine motor pool threshold than is the EMG due to its linearity. In fact, when two lines are fitted to the data, they intersect at the threshold. For some of the subjects however, EMG processing was able to detect a threshold at a lower stimulus than was discernable on the force trace. This apparent dichotomy is likely due to the limited resolution of the load cell that was used. The use of a more sensitive load cell can be implemented to resolve this.

The data in FIG. 4 shows that the tap force delivered on the tendon was, for the most part, the same for the spastic limb as for the control for each subject. This implies that the muscle spindle receptors of the different muscles were stretched equivalently, consistent with previous work (see, e.g., Lee W A, Boughton A, Rymer W Z, Absence of stretch reflex gain enhancement in voluntarily activated spastic muscle, Exp Neurol. 1987 November 98(2):317-35).

Therefore, the input to the motor neuron pool for both sides was essentially the same. The spindle afferent information elicited by a tap can be viewed as being proportional to the force delivered on the tendon.

It was also shown that spindle gamma drive is not affected by stroke (Edin, B B, Vallbo, A B, Dynamic response of human muscle spindle afferents to stretch, Journal of neurophysiology, 1990). This also supports that the mechanical perturbation on the tendon would be the same on both sides.

As discussed above, these data suggest that the threshold of the motor neuron pool declined after a hemiparetic stroke, as described in Katz R T and Rymer R W, Spastic hypertonia: mechanisms and measurement, Arch Phys Med Rehabil. 1989 February; 70(2):144-55; and Powers R K, Campbell D L, Rymer W Z, Stretch reflex dynamics in spastic elbow flexor muscles, Ann Neurol. 1989 January; 25(1):32-42. Using this analysis, it is possible to estimate a value for the change in the threshold, expressed as a difference in length or in tendon stress.

In sum, three significant results can be extracted from the above. The first is it is possible to deliver and measure a reflex response of a single muscle with a small fully embedded tool (actuator and sensor). Second, the force trace results show a linear relationship between tap force and maximum reflex force. Third, a simple method is provided to estimate the threshold of a motoneuron pool.

The techniques described above can measure threshold, gain, and intrinsic properties of the muscle. In a potential application, one or more of these values could be tracked throughout a patient's life to establish a historical database for this person. This could permit determining if something was going wrong at the cortex level before the normal clinical signs (hypertonia, defective reflexes, etc.). Additionally, this technique may be used to detect the onset of ALS, MSA, mini strokes, muscle atrophy, demyelinating diseases, and Parkinson's. This could also be used in simple tests such as conduction velocity.

The values measured could further be used in dosage of drugs for patients where one could measure the effects a drug might have on treating spasticity. In another embodiment, a proper dosage of medicine, such as Botox, could be established based on the above-described measures of spasticity or passive muscle properties. Finally these measurements and techniques via the device described above could also be used in surgical procedures where muscle activity can be measured, for example, where a patient gets skeletal muscle replacement surgeries. These values could be used to measure the success of such a procedure. Additionally, these values and techniques could be utilized in the veterinary field to make determinations of ailments that are difficult to diagnose given a lack of ability to communicate. Without a historical database for a patient, the values are determined vs the contralateral limb. With the availability of the historical patient database, the values can be measured against the patient's own historical values. However, with a many-patient database, it may be possible to make statistical inferences and classifications that could permit an analysis based on the population as a whole or certain groups or classifications determined from the database, although between patient variances may make certain inferences less certain. Given enough data samples, attributes such as muscle type, patient size, age, etc. could be stored and used as a comparison basis as well.

### Embodiment 2—Passive Muscle Properties

In addition to threshold detection, progressive indentation of the tendon can also be used to predict intrinsic mechanical properties of muscles. According to another embodiment of the invention, a method for diagnosing a muscle condition can be determined based on a stiffness of perturbed tendons. The measured stiffness of perturbed tendons is generally greater than that of normal tendons, and the instantaneous force elicited by a tendon tap is also increased. Both of these features suggest systematic changes in neuromuscular mechanical properties, discernable at the level of the peripheral tendon and muscles. Such a stiffness measurement can be used in accordance with various embodiments of the invention in assessing prognoses of stroke survivors, and in recommending targeted therapeutic interventions designed to address the specific deficits present in each patient.

In the course of testing for increased reflex responses as described above, a cardinal feature of spastic hypertonia, the intrinsic stiffness of the muscle is often increased, and the transient response to a tendon tap is also increased. Thus, the passive properties of muscle are also changed in parallel with the neurological abnormalities. These features can be recognized using the actuator described above to progressively load the tendon. An embodiment of the invention, described below, demonstrates these principles.

### Experimental Setup

FIG. 1, as described above and again partially referenced in this section, illustrates the experimental embodiment shown in which a series of controlled taps were applied to a tendon of the biceps brachii with a linear actuator. FIG. 1 illustrates the position-controlled linear actuator placed perpendicular to the tendon of the biceps brachii. Referring to FIG. 2B, the muscle was immobilized S102, the actuator was placed over the tendon S104 and lowered onto the tendon sequentially S106′, S118′ relative to the skin and then used to tap the tendon S110, S124 according to a tap sequence described in more detail below. Force measurements were taken from the tendon S112′, S126. These measurements provided a benchmark for the reflex threshold estimations.

In two stroke survivors, force measurements were obtained for five successive tendon pulses applied at each of approximately twenty tendon indentation positions. As above, tendon indentation was referenced to the skin surface, and the responses were compared between impaired and contralateral limbs.

The arm position was optimized such that the elbow and shoulder joint angles ensured a maximal reflex response from the biceps. The subjects had approximately 45° wrist supination, a 120° elbow extension, 45° shoulder abduction, and a 10° shoulder flexion. The subjects were placed as closely as possible to the ideal joint angles which were recorded to ensure repeatability on the contralateral side.

The position controlled linear actuator 10 was mounted on the mechanical frame directly above the patients' tendon 106. The linear actuator was placed at an angle of approximately 90° to the tendon and, for recording purposes, the position was zeroed at the surface of the skin. The tapper tip 14 was then methodically lowered in increments of 1 mm, using a micrometer 18 attached to the actuator 10. The position of the linear actuator 10 was constantly monitored, guaranteeing repeatability in the stimuli and in the measurements of the reflex. In addition, a high precision force sensor 16 in the form of a load cell was attached to the tapper head which enabled measuring the tension in the tendon 106.

At each tendon indentation, each of which constituted a new trial, a pulse sequence of five transient 1 mm taps were applied to the tendon every two seconds. The taps were administered over a distance of up to 25 mm indentation into the tendon at 1 mm intervals. The controllability of the linear actuator ensured the repeatability of the stimuli over duration of each experimental session. Additionally, since the actuator 10 was placed to pre-stretch the tendon 106, constant contact between the tendon 106 and the actuator 10 was ensured.

As noted above with regard to FIG. 3A, the graph shows a typical force trace versus time. Three different aspects of this trace are considered: the preload, the maximum tap force and the maximum reflex force. These provide the force at which the tendon is stretched at (preload), the force at which the tendon was tapped at (maximum tap force), and the maximum force generated by the muscle after the tap (maximum reflex force). Stroke participants were adults who had sustained a hemiparetic stroke at least six months prior to experimental testing.

### Preload Force Measurement

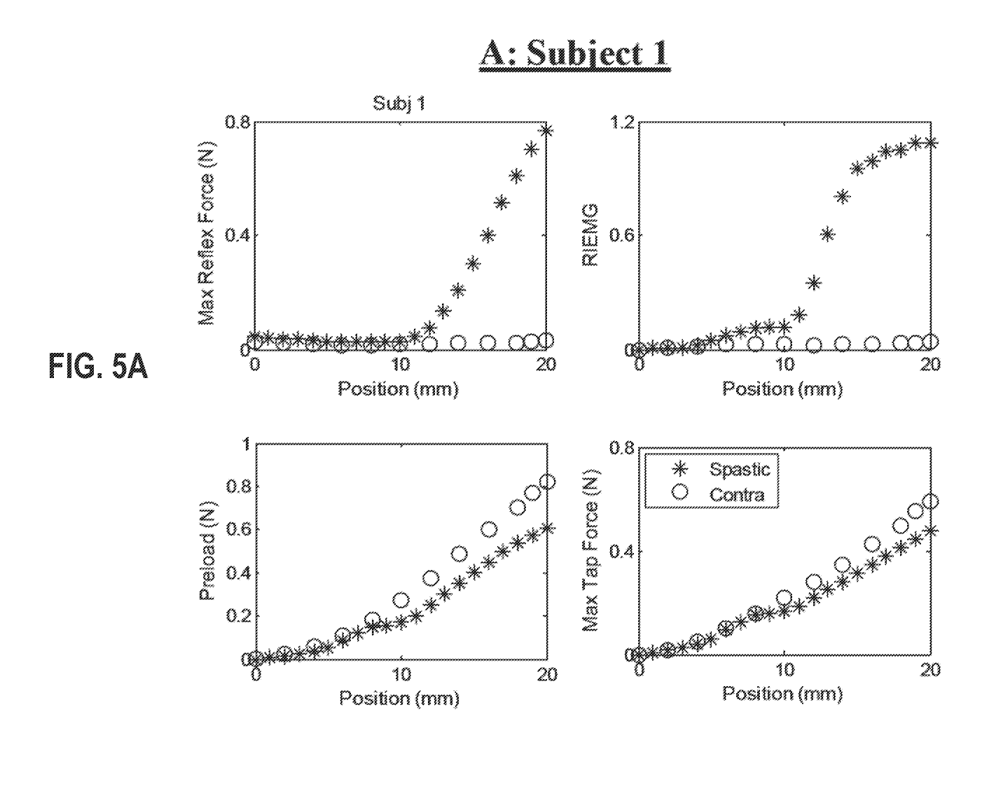

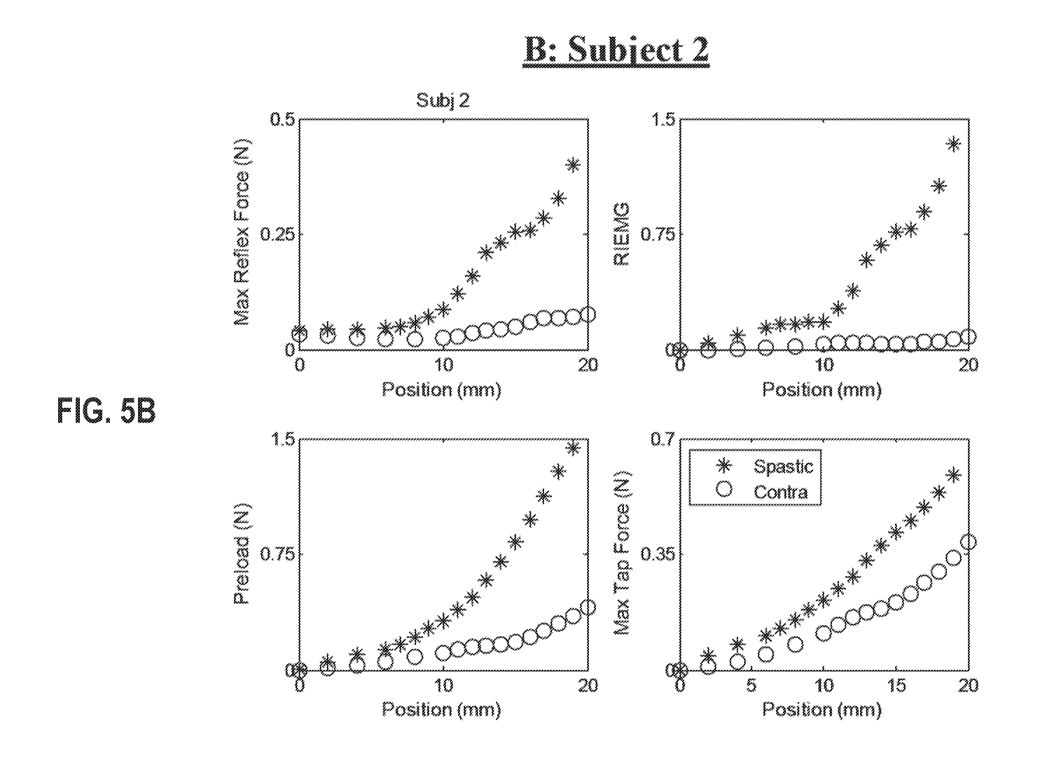

FIGS. 5A and 5B plot both the preload force and the maximum tap force versus position for both subjects. The preload force is the steady state force that the tendon reaches at a new indentation position. Hence plotting preload versus position gives, via the slope, the stiffness of the muscle. The data from the preload plot of Subject 1 (FIG. 5A), illustrates that the stiffness of the spastic muscle and the contralateral muscle are relatively similar suggesting that they had similar intrinsic mechanical properties. On the other hand, Subject 2 exhibited a much steeper slope between static force and tendon indentation on the spastic side, as compared to the contralateral counterpart (FIG. 5B). This corresponds to changes in intrinsic muscle properties in parallel with neurological impairment, since both subjects exhibited hyperreflexia as characterized by a significantly lower reflex threshold as seen in plots of EMG and force vs. position, although these changes do not appear to be present in every case.

### Maximum Tap Force Measurements

Plotting maximum tap force versus indentation position gives another measure of muscle stiffness. In this case, it is the rate of change of stiffness as the indentation deepens. The maximum tap force Fₜₐₚ (see FIG. 3A) is measured prior to the reflex response time window and thus only measures the passive properties. Since the maximum tap force Fₜₐₚ is calculated relative to the preload force Fᵢ, it should be understood as the force that is measured relative to a 1 mm displacement during the tap sequence. Hence each value on the plot can be interpreted as a local stiffness for that indentation. As these values are plotted against the indentation position, the resulting plot represents the rate of change of stiffness between each indentation.

In some cases, such as for Subject 2, there is a progressive increase in the baseline (or preload force) as the tendon is indented progressively. This increase in background force, relative to the contralateral side, suggests that the muscle tendon complex may have changed its mechanical properties as a result of the stroke, and that this increase in force is accompanied by an increase in incremental stiffness, appearing as a higher tapping force.

In contrast, for some subjects (as in Subject 1), the background (or preload) force does not increase with increasing tendon indentation, and there may be no associated increase in tapping force either. These latter types of cases do not appear to display any global changes in intrinsic mechanical properties of the spastic muscle.

Severe prolonged spasticity may be associated with increases in intrinsic muscle stiffness, but this is not a constant. One possible mechanism that may explain the passive force changes is that has been a progressive muscle fiber shortening, associated with shortening of the tendon as well. In other words, the length tension of the muscle tendon complex is shifted to the left, with a change on the rest length of the spring “analog”. It is known that many spastic muscles undergo contracture, which may also include global changes in stiffness. With a broader indentation range, one may be able to distinguish between muscle fiber shortening and global changes in intrinsic stiffness. Lieber, e.g., (Surgical Myometer Method, U.S. Pat. No. 4,570,641, have disclosed in vivo measurement of human wrist extensor muscle sarcomere length changes J of Neurophysiology, http://www-neuromus.ucsd.edu/more_html/overview.shtml) have described changes in muscle architecture in children with CP (spasticity is a resultant effect), and has provided work on stroke patients. His findings were that muscle had a larger ratio between connective tissue to muscle fiber. Connective tissue is much stiffer than muscle fiber and does not show any electrical activity. Hence when one measures a higher passive stiffness and low EMG activity, one can infer that the muscle has changed and the connective tissue to muscle fiber ratio has gone up.

### Other Variations

The tendon tapper technique described herein provides useful information about reflex pathways, including those mediating spasticity, and it also can provide valuable information about other potentially useful mechanical properties of spastic muscle. Additionally, aspects separately describing distinctions in measurements for the following have been discussed above: 1) force measurement at tendon vs. EMG measurement at muscle; 2) threshold response vs. gain; 3) comparative measures for spastic and contralateral sides vs. for older and current measured data (per subject or across subjects); and 4) spasticity vs. passive muscle characteristics. However, the present invention is not limited to such distinctions in isolation—the measurement system and method can combine any/all of these aspects in various embodiments.

Although the tapper position above has been described above with respect to indentation from the skin surface (at the point of initial tapper contact), the tapper position can also be measured and recorded with respect to a bone surface by using ultrasound or ultrasonic techniques. It may also be possible to measure the tapper position with respect to the tendon, although movement of the tendon may make such measurements difficult or inaccurate.

The indentation of the tapper has been described as being step-by-step, incrementally. However, the tapper could be indented at different indentation values than fixed increments. One could also use some form of a method in which the threshold value is approached from either side by smaller and smaller increments in order to pinpont a precise value. Additionally, for more precision, resolutions finer than one mm could be utilized in a general region in which spasticity is determined.

The system or systems described herein may be implemented on any form of computer or computers and the components may be implemented as dedicated applications or in client-server architectures, including a web-based architecture, and can include functional programs, codes, and code segments. Any of the computers may comprise a processor, a memory for storing program data and executing it, a permanent storage such as a disk drive, a communications port for handling communications with external devices, and user interface devices, including a display, keyboard, mouse, etc. When software modules are involved, these software modules may be stored as program instructions or computer readable codes executable on the processor on a computer-readable media such as read-only memory (ROM), random-access memory (RAM), CD-ROMs, magnetic tapes, floppy disks, and optical data storage devices. The computer readable recording medium can also be distributed over network coupled computer systems so that the computer readable code is stored and executed in a distributed fashion. This media can be read by the computer, stored in the memory, and executed by the processor.

All references, including publications, patent applications, and patents, cited herein are hereby incorporated by reference to the same extent as if each reference were individually and specifically incorporated by reference and set forth in their entirety herein.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The present invention may be described in terms of functional block components and various processing steps. Such functional blocks may be realized by any number of hardware and/or software components configured to perform the specified functions. For example, the present invention may employ various integrated circuit components, e.g., memory elements, processing elements, logic elements, look-up tables, and the like, which may carry out a variety of functions under the control of one or more microprocessors or other control devices. Similarly, where the elements of the present invention are implemented using software programming or software elements the invention may be implemented with any programming or scripting language such as C, C++, Java, assembler, or the like, with the various algorithms being implemented with any combination of data structures, objects, processes, routines or other programming elements. Functional aspects may be implemented in algorithms that execute on one or more processors. Furthermore, the present invention could employ any number of conventional techniques for electronics configuration, signal processing and/or control, data processing and the like. The words “mechanism” and “element” are used broadly and are not limited to mechanical or physical embodiments, but can include software routines in conjunction with processors, etc.

The particular implementations shown and described herein are illustrative examples of the invention and are not intended to otherwise limit the scope of the invention in any way. For the sake of brevity, conventional electronics, control systems, software development and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”.

The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The use of the terms “a” and “an” and “the” and similar referents in the context of describing the invention (especially in the context of the following claims) are to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein. Finally, the steps of all methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. Numerous modifications and adaptations will be readily apparent to those skilled in this art without departing from the spirit and scope of the present invention.

What is claimed is:

1. A method for measuring a spasticity of a muscle, comprising:
a) generally immobilizing a muscle;
b) placing a tapping element at a skin position over a tendon associated with the muscle to establish a tap point;
c) moving the tapping element to an indentation position at a predetermined distance from a reference point;
d) at the indentation position, measuring a preload force on the tapping element and then providing one or more taps in a tap group at the tap point;
e) measuring a force curve on the tapping element during a predefined reflex response time window after providing each said tap;
f) determining and recording, for each said tap, a tap response value based upon a predefined tap criteria associated with the force curve;
g) determining, for each said tap group, whether or not a spastic response is present based upon a predefined tap group criteria associated with the tap response values within at least one of a current or previous tap group;
h1) if no spastic response is determined to be present, then moving the tapping element to a new indentation position and repeating steps (d)-(g); and
h2) if a spastic response is determined to be present, then establishing at least one of the indentation position and the preload force as the determined muscle spasticity threshold.

2. The method of claim 1, wherein the new indentation position is the predetermined distance.

3. The method of claim 2, wherein the predetermined distance is 1 mm.

4. The method of claim 1, wherein the predetermined distance is 1 mm.

5. The method of claim 1, wherein the tap group consists of five taps.

6. The method of claim 1, wherein the predefined criteria is selected from the group consisting of:
an average or median value of the indentation reflex forces of the tap group;
a minimum value of the indentation reflex forces of the tap group; and
a maximum value of the indentation reflex forces of the tap group.

7. The method of claim 1, wherein the reflex response time window begins at 20 ms from a tap start.

8. The method of claim 7, wherein the reflex response time window ends at 1 s from the tap start.

9. The method of claim 1, further comprising:
after step (h2), moving the tapping element to a new indentation position and repeating steps (d)-(g); and
establishing a gain value that represents a change in a first variable selected from the group consisting of indentation position, preload force, tap distance, and tap force, and a second non-identical variable selected from the same group.

10. The method of claim 1, further comprising:
attaching an EMG electrode to a skin surface over a portion of the muscle prior to tapping the tendon;
at step (e), measuring an EMG signal curve during an EMG reflex response time window;
at step (f), determining an average rectified EMG signal (RIEMG) from the EMG signal curve; and
i) establishing a second determined muscle spasticity threshold at an indentation position at which RIEMG is greater than or equal to a predefined threshold average rectified EMG signal value.

11. A method for making a medical diagnosis, comprising:
performing the method of claim 1 on both spastic and contralateral sides of a subject to obtain a spastic side muscle spasticity threshold and a contralateral side muscle spasticity threshold; and
establishing the medical diagnosis based on a difference between the spastic side muscle spasticity threshold and the contralateral side muscle spasticity threshold.

12. The method according to claim 11, wherein the medical diagnosis is selected from the group consisting of hypertonia, ALS, MSA, mini strokes, muscle atrophy, demyelinating diseases, and Parkinson's.

13. A method for making a medical diagnosis, comprising:
performing the method of claim 1 at a first subject office visit and at a second subject office visit at a different point in time, and establishing a respective first muscle spasticity threshold and second muscle spasticity threshold; and
establishing the medical diagnosis based on a difference between the first muscle spasticity threshold and the second muscle spasticity threshold.

14. The method according to claim 13, wherein the medical diagnosis is selected from the group consisting of hypertonia, ALS, MSA, mini strokes, muscle atrophy, demyelinating diseases, and Parkinson's.

15. The method according to claim 1, further comprising:
repeating steps (a)-(h2) for a plurality of subject muscles; and
storing the determined muscle spasticity threshold value in a database along with at least one of subject, muscle, and date/time identifying attributes associated with the stored value.

16. The method according to claim 1, wherein the tap response value is determined to be a “0” if a maximum value of the force curve is less than a predefined force curve criteria, and is determined to be a “1” if the maximum value of the force curve is greater than or equal to the predefined force curve criteria.

17. The method according to claim 16, wherein the predefined force curve criteria is a mean value of the preload force plus three times the standard deviation of the preload force.

18. The method according to claim 1, wherein a number of taps in a tap group is an odd number that is three or greater.

19. The method according to claim 1, wherein:
the tap response value is determined to be a “0” if a maximum value of the force curve is less than a predefined force curve criteria, and is determined to be a “1” if the maximum value of the force curve is greater than or equal to the predefined force curve criteria;
a number of taps in a tap group is an odd number that is three or greater; and
a spastic response is determined to be present according to the predefined tap group criteria of a majority of tap response values within the tap group is “1”.

20. The method according to claim 1, wherein the reference point is a skin surface in an initial no-load state of the tapper.

21. The method according to claim 1, wherein the reference point is a bone surface below the tapper.

22. A method for measuring a stiffness of a muscle, comprising:
a) generally immobilizing a muscle;
b) placing a tapping element at a skin position over a tendon associated with the muscle to establish a tap point;
c) moving the tapping element to a first indentation position at a predetermined distance from a reference point;
d) at the indentation position, measuring a preload force on the tapping element and then providing one or more taps at the tap point;
e) measuring forces on the tapping element prior to a reflex response time window after providing the tap(s) and determining a maximum value that is a maximum tapping force value;
f) moving to a second indentation position, and repeating steps (d) and (e); and
g) calculating a muscle stiffness value based on a line slope at at least one of the first and second indentation positions based on the maximum tapping force value at the respective indentation position.
